(19) United States
(12) Patent Application Publication
(10) Pub. No.: US 2025/0279178 A1
(43) Pub. Date: Sep. 4, 2025

(54) MEDICAL IMAGE PROCESSING APPARATUS, MEDICAL IMAGE PROCESSING METHOD AND NON-TRANSITORY COMPUTER-READABLE STORAGE MEDIUM

(71) Applicant: CANON MEDICAL SYSTEMS CORPORATION, Otawara-shi (JP)

(72) Inventors: Gakuto AOYAMA, Kawasaki (JP); Hironaga NOGUCHI, Saitama (JP); Junichi OOIDA, Ota (JP)

(73) Assignee: CANON MEDICAL SYSTEMS CORPORATION, Otawara-shi (JP)

(21) Appl. No.: 19/058,794

(22) Filed: Feb. 20, 2025

(30) Foreign Application Priority Data
Feb. 29, 2024 (JP) 2024-029447
Nov. 20, 2024 (JP) 2024-202111

(51) Int. Cl. G16H 20/40; G06T 7/00

(52) U.S. Cl. CPC G16H 20/40; G06T 7/0012; G06T 2207/30048

(57) ABSTRACT

A medical image processing apparatus according to an embodiment includes processing circuitry. The processing circuitry obtains an organ model, in which the target organ for treatment is expressed, based on a medical image; identifies a treatment condition which represents a treatment-related condition including at least the treatment position in the organ model; estimates a post-treatment feature quantity based on the organ model and the treatment condition; decides on a display condition regarding the treatment position in the organ model based on the estimated feature quantity; and displays, in a display device, the organ model according to the decided display condition.

### CROSS-REFERENCE TO RELATED APPLICATIONS

This application is based upon and claims the benefit of priority from Japanese Patent Application No. 2024-029447, filed on Feb. 29, 2024 Japanese Patent Application No. 2024-202111, filed on Nov. 20, 2024, the entire contents of which are incorporated herein by reference.

### FIELD

Embodiments described herein relate generally to a medical image processing apparatus, a medical image processing method and a non-transitory computer-readable storage medium.

### BACKGROUND

Conventionally, various treatment methods have been proposed for treating heart disease. For example, as a treatment method for treating the disease related to the heart valves such as the mitral valve, a treatment method is known that is based on a heart valve repairing device in which a catheter is used.

As a heart valve repairing device, for example, a mitral valve repairing device is known that is used in treating mitral regurgitation (MR). A mitral valve repairing device is used in a procedure called edge-to-edge repair that is meant for increasing the synapsed region by grasping the anterior leaflet and the posterior leaflet of the mitral valve.

For example, in the case of performing treatment using a mitral valve repairing device, before starting the actual treatment, sometimes an estimation operation is performed in which, regarding a plurality of treatment conditions (for example, the grasping positions of a plurality of mitral valve repairing devices), a post-treatment feature quantity (for example, the valve area or the volume of regurgitant flow) is estimated that enables estimation of the effect of treatment in the case of performing the treatment under each condition.

### BRIEF DESCRIPTION OF THE DRAWINGS

FIG. 1 is a block diagram illustrating an exemplary configuration of a medical information processing system according to an embodiment;

FIG. 2 is a block diagram illustrating an exemplary configuration of an X-ray CT apparatus according to the embodiment;

FIG. 3 is a diagram illustrating an exemplary definition of an organ model (a mitral valve mesh) according to the embodiment;

FIG. 4 is a diagram illustrating another exemplary definition of the organ model (the mitral valve mesh) according to the embodiment;

FIG. 5 is a flowchart for explaining an example of the operations performed in the medical image processing apparatus according to the embodiment;

FIGS. 6 and 7 are diagrams for explaining an example of a setting operation for setting the treatment position;

FIG. 8 is a diagram for explaining an example of a deciding operation for deciding on the display conditions according to the embodiment;

FIG. 9 is a diagram for explaining an example of an addition setting operation for adding a treatment position according to the embodiment;

FIG. 10 is a diagram for example of an updating operation for updating the display conditions according to the embodiment; and

FIGS. 11 to 13 are diagrams for explaining an example of setting the display conditions of the organ model according to a second modification example.

### DETAILED DESCRIPTION

A medical image processing apparatus according to an embodiment includes processing circuitry. The processing circuitry obtains an organ model, in which the target organ for treatment is expressed, based on a medical image; identifies a treatment condition which represents a treatment-related condition including at least the treatment position in the organ model; estimates a post-treatment feature quantity based on the organ model and the treatment condition; decides on a display condition regarding the treatment position in the organ model based on the estimated feature quantity; and displays, in a display device, the organ model according to the decided display condition.

An exemplary embodiment of a medical image processing apparatus and a medical image processing method is described below in detail with reference to the accompanying drawings.

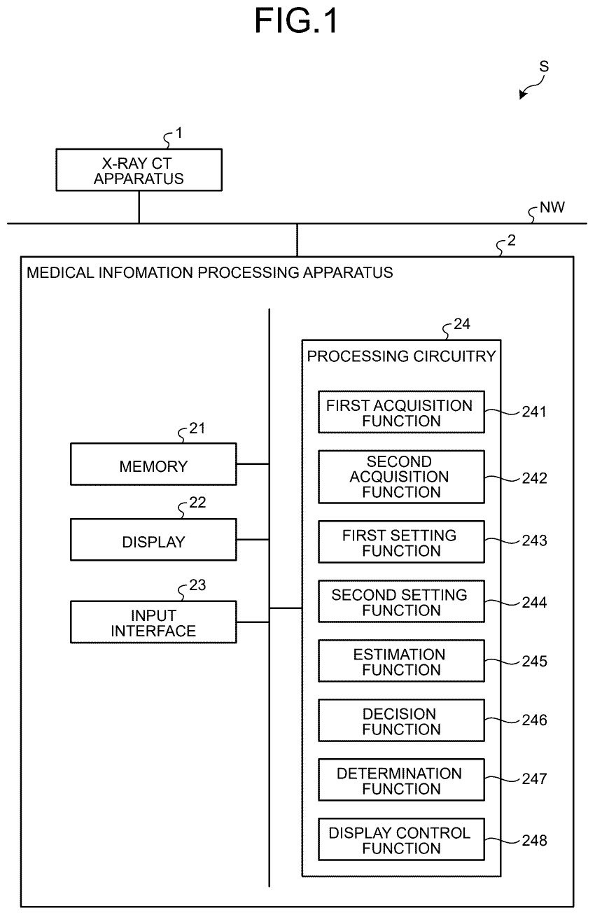

In the embodiment, as illustrated in FIG. 1, the explanation is given about a medical information processing system S that includes an X-ray CT apparatus 1 and a medical image processing apparatus 2. FIG. 1 is a block diagram illustrating an exemplary configuration of the medical information processing system S according to the embodiment. In the embodiment, it is assumed that the operations (explained later) are performed based on projection data collected by the X-ray CT apparatus 1 illustrated in FIG. 1. The X-ray CT apparatus 1 and the medical image processing apparatus 2 are connected to each other via a network NW.

As long as the connection can be established via the network NW, the X-ray CT apparatus 1 and the medical image processing apparatus 2 can be installed at arbitrary installation positions. For example, the X-ray CT apparatus 1 and the medical image processing apparatus 2 can be installed in mutually different facilities. Thus, the network NW can be configured using a closed-type local network between the facilities, or can be a network such as the Internet.

The communication between the X-ray CT apparatus 1 and the medical image processing apparatus 2 either can be performed via another device such as an image archiving apparatus or can be performed directly without involving any other device. Examples of the image archiving device include a PACS server (PACS stands for Picture Archiving and Communication System).

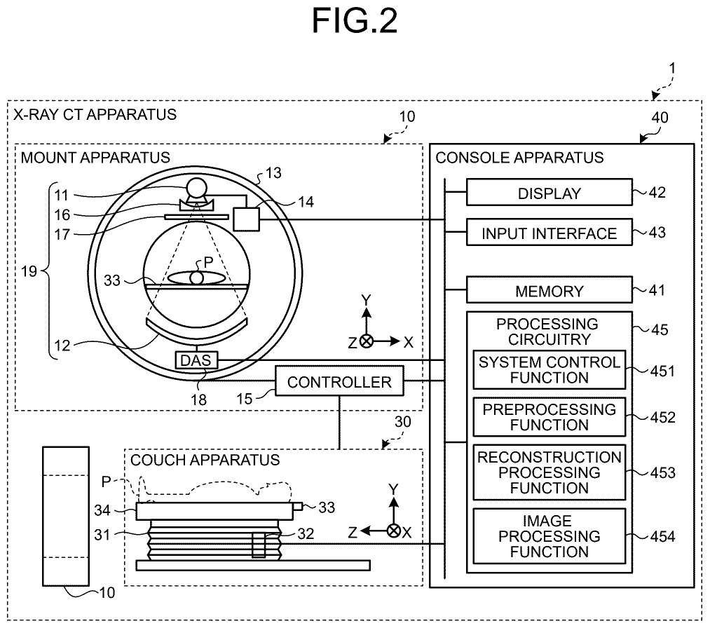

Firstly, explained below with reference to FIG. 2 is the X-ray CT apparatus 1. FIG. 2 is a block diagram illustrating an exemplary configuration of the X-ray CT apparatus 1 according to the embodiment. As illustrated in FIG. 2, the X-ray CT apparatus 1 includes a mount apparatus 10, a couch apparatus 30, and a console apparatus 40.

In the present embodiment, the longitudinal direction of the rotation axis of a rotatable frame 13 in the non-tilted state is defined as the Z-axis direction; the direction that is orthogonal to the Z-axis direction and that is oriented from the center of rotation toward a columnar support meant for supporting the rotatable frame 13 is defined as the X-axis; and the direction that is orthogonal to the Z-axis and the X-axis is defined as the Y-axis.

The mount apparatus 10 includes an imaging system 19 for taking medical images that are to be used in making a diagnosis. The imaging system 19 is configured with, for example, an X-ray tube 11, an X-ray detector 12, a wedge 16, and a collimator 17. Thus, the mount apparatus 10 includes the imaging system 19 that bombards X-rays onto a subject P and collects projection data from the detection data about the X-rays that have passed through the subject P.

The mount apparatus 10 has an opening portion formed to accommodate the subject P. A couchtop 33 on which the subject P is asked to lie down is housed inside the opening portion, with the side on which the couch apparatus 30 is disposed serving as the entry side.

The mount apparatus 10 includes the X-ray tube 11, the wedge 16, the collimator 17, the X-ray detector 12, an X-ray high-voltage generator 14, a data acquisition system (DAS) 18, the rotatable frame 13, a controller 15, and the couch apparatus 30.

The X-ray tube 11 is a vacuum tube that receives the application of a high voltage from the X-ray high-voltage generator 14 and that generates X-rays by bombarding thermal electrons from a cathode (filament) onto an anode (target). For example, the X-ray tube 11 is a rotating anode X-ray tube that generates X-rays by bombarding thermal electrons onto a rotating anode.

The wedge 16 is a filter for adjusting the X-ray dosage of the X-rays that are bombarded from the X-ray tube 11. More particularly, the wedge 16 is a filter that transmits and attenuates the X-rays, which are bombarded from the X-ray tube 11, in such a way that the X-rays bombarded from the X-ray tube 11 onto the subject P have a predetermined distribution.

For example, the wedge 16 is a wedge filter or a bow-tie filter, and is manufactured by processing aluminum to achieve a predetermined target angle and a predetermined thickness.

The collimator 17 is a lead plate meant for limiting the X-rays, which have passed through the wedge 16, within an X-ray bombardment range; and constitutes a slit due to a combination of a plurality of lead plates. The collimator 17 is sometimes also called an X-ray limiter.

The X-ray detector 12 detects the X-rays that were bombarded from the X-ray tube 11 and that have passed through the subject P; and outputs, to the DAS 18, an electrical signal corresponding to the X-ray dosage. The X-ray detector 12 includes, for example, a plurality of detection element arrays in each of which a plurality of X-ray detection elements is arranged in the channel direction along a single circular arc centered on the focal point of the X-ray tube 11. The channel direction implies the circumferential direction of the rotatable frame 13.

The X-ray detector 12 includes, for example, a plurality of detection element arrays in each of which a plurality of X-ray detection elements is arranged in the channel direction along a single circular arc centered on the focal point of the X-ray tube 11. For example, the X-ray detector 12 has a structure in which a plurality of X-ray detection element arrays, in each of which a plurality of X-ray detection elements is arranged in the channel direction, is arranged along the slice direction (also called the body axis direction or the row direction).

Moreover, the X-ray detector 12 is, for example, an indirect-transform-type detector that includes a grid, a scintillator array, and an optical sensor array. The scintillator array includes a plurality of scintillators, each of which includes a scintillator crystal that outputs a light having the photon quantity according to the incident X-ray dosage. The grid is disposed on that face of the scintillator array which is on the X-ray incidence side, and includes an X-ray shield plate equipped to absorb the scattered X-rays.

The optical sensor array has the function of converting the light from the scintillator into electric signals according to the light intensity; and includes an optical sensor such as a photomultiplier tube (PMT). Meanwhile, alternatively, the X-ray detector 12 can be a direct-transform-type detector that includes a semiconductor element for converting the incident X-rays into electrical signals.

The X-ray high-voltage generator 14 includes electrical circuitry such as a transformer and a rectifier, and also includes a high-voltage generator having the function of generating a high voltage to be applied onto the X-ray tube 11 and an X-ray controller for controlling the output voltage according to the X-rays bombarded from the X-ray tube 11. The high-voltage generator can be of the transformer type or the inverter type.

The X-ray high-voltage generator 14 either can be disposed in the rotatable frame 13, or can be disposed in the fixed frame (not illustrated) of the mount apparatus 10. The fixed frame is used in rotatably supporting the rotatable frame 13.

The DAS 18 includes an amplifier for performing amplification with respect to the electrical signals output from each X-ray detection element of the X-ray detector 12 and includes an A/D converter for converting the amplified electrical signals into digital signals; and generates detection data. The detection data generated by the DAS 18 is transferred to the console apparatus 40. The detection data is, for example, in the form of a sinogram.

A sinogram represents data in which, the projection data generated for each position of the X-ray tube 11 (hereinafter, also called the view angle) and for each X-ray detection element is illustrated in a corresponding manner to the view direction and the channel direction. The view direction corresponds to the view angle and implies the X-ray bombardment direction.

When one-time scanning is performed using only a single detection element array in the X-ray detector 12, it becomes possible to generate a single sinogram for a single instance of scanning. When helical scanning or volume scanning is performed using a plurality of detection element arrays in the X-ray detector 12, it becomes possible to generate a plurality of sinograms for a single instance of scanning.

The rotatable frame 13 is an annular frame that supports the X-ray tube 11 and the X-ray detector 12 opposite to each other and that rotates the X-ray tube 11 and the X-ray detector 12 under the control of the controller 15. Meanwhile, in addition to supporting the X-ray tube 11 and the X-ray detector 12, the rotatable frame 13 also supports the X-ray high-voltage generator 14 and the DAS 18.

The rotatable frame 13 is rotatably supported by the non-rotatable portion of the mount apparatus 10 (for example, supported by a fixed frame (not illustrated in FIG. 2)). Herein, the rotation mechanism includes, for example, a motor that generates a rotary driving force and a bearing that transmits the rotary driving force to the rotatable frame 13 and causes the rotatable frame 13 to rotate. The motor is disposed in, for example, the non-rotatable portion. The bearing is physically connected to the rotatable frame 13 and the motor, and causes the rotatable frame 13 to rotate according to the torque of the motor.

In the rotatable frame 13 as well as in the non-rotatable portion, communication circuitry is disposed that is either of the contactless type or of the contact type. As a result, the units that are supported by the rotatable frame 13 can communicate with the non-rotatable portion or with external apparatuses of the mount apparatus 10.

For example, when optical communication is implemented as the contactless communication method, the detection data generated by the DAS 18 is sent using optical communication from a transmitter, which is disposed in the rotatable frame 13 and which includes a light emitting diode (LED), to a receiver, which is disposed in the non-rotatable portion of the mount apparatus 10 and which includes a photodiode. Moreover, the detection data is transferred from the non-rotatable portion to the console apparatus 40 using the transmitter.

Meanwhile, as the communication method, it is possible to implement contactless data transfer using the capacitive coupling method or the radio wave method, or it is possible to implement a contact-type data transmission method using a slip ring and an electrode brush.

The controller 15 includes processing circuitry having a central processing unit (CPU), and includes a driving mechanism including a motor and an actuator. The controller 15 receives an input signal from an input interface that is attached to the console apparatus 40 or the mount apparatus 10, and accordingly controls the operations of the mount apparatus 10 and the couch apparatus 30.

For example, the controller 15 receives an input signal and performs control to rotate the rotatable frame 13, or performs control to tilt the mount apparatus 10, or performs control to operate the couch apparatus 30 and the couchtop 33. Regarding the control for tilting the mount apparatus 10, according to inclination angle (tilt angle) information input via the input interface attached to the mount apparatus 10, the controller 15 rotates the rotatable frame 13 around the axis parallel to the X-axis direction.

Meanwhile, the controller 15 either can be disposed in the mount apparatus 10, or can be disposed in the console apparatus 40.

The couch apparatus 30 is an apparatus on which the subject P, who is the target for scanning, is made to lie down and is moved. The couch apparatus 30 includes a base 31, a couch driving apparatus 32, the couchtop 33, and a supporting frame 34. The base 31 is a housing that supports the supporting frame 34 to be movable in the vertical direction. The couch driving apparatus 32 is a motor or an actuator that moves the couchtop 33, on which the subject P is lying down, in the long-axis direction of the couchtop 33 (in FIG. 2, the Z-axis direction).

The couchtop 33 is a plate placed on the upper surface of the supporting frame 34, and the subject P is asked to lie down on the couchtop 33. Meanwhile, in addition to moving the couchtop 33, the couch driving apparatus 32 can also move the supporting frame 34 in the long-axis direction of the couchtop 33.

The couch driving apparatus 32 moves the base 31 in the vertical direction according to a control signal from the controller 15. Moreover, the couch driving apparatus 32 moves the couchtop 33 in the long-axis direction (the Z-axis direction) according to a control signal from the controller 15.

The console apparatus 40 receives an operation performed by the operator with respect to the X-ray CT apparatus 1, and reconstructs the X-ray CT image data from the X-ray detection data collected by the mount apparatus 10. The console apparatus 40 includes a memory 41, a display 42, an input interface 43, and processing circuitry 45.

The memory 41 is implemented, for example, using a semiconductor memory device such as a random access memory (RAM) or a flash memory, or using a hard disk, or using an optical disc. The memory 41 is used to store, for example, projection data and reconstructed image data. Moreover, the memory 41 is used to store the imaging protocol.

In the imaging protocol, a procedure for controlling the imaging system 19 and taking images of the subject P is specified. For example, the imaging protocol represents a group of parameters such as the body part to be subjected to imaging, the imaging conditions, the imaging range, the reconstruction conditions, the operation of the mount apparatus 10 (the imaging system 19), and the operation of the couch apparatus 30.

The memory 41 is used to store dedicated computer programs meant for implementing a system control function 451 (explained later), a preprocessing function 452 (explained later), a reconstruction processing function 453 (explained later), and an image processing function 454 (explained later).

The display 42 is a monitor referred to by the operator and is used to display a variety of information. For example, the display 42 is used to output medical images (CT images) generated by the processing circuitry 45, and to output a graphical user interface (GUI) meant for receiving various operations from the operator. Examples of the display 42 include a liquid crystal display or a cathode ray tube (CRT) display.

The input interface 43 receives various input operations from the operator, converts the input operations into electrical signals, and outputs the electrical signals to the processing circuitry 45. For example, from the operator, the input interface 43 receives collection conditions to be implemented at the time of collecting the projection data, or receives reconstruction conditions to be implemented at the time of reconstructing the CT image data, or receives image processing conditions to be implemented at the time of generating a postprocessing image from a CT image.

The input interface 43 is implemented using, for example, a mouse, a keyboard, a trackball, switches, buttons, or a joystick. Meanwhile, the input interface 43 can alternatively be disposed in the mount apparatus 10. Still alternatively, the input interface 43 can be configured using a tablet terminal capable of performing wireless communication with the console apparatus 40.

The processing circuitry 45 controls the operations of the entire X-ray CT apparatus 1. For example, the processing circuitry 45 includes the system control function 451, the preprocessing function 452, the reconstruction processing function 453, and the image processing function 454.

In the embodiment, the processing functions implemented by the constituent elements, namely, the system control function 451, the preprocessing function 452, the reconstruction processing function 453, and the image processing function 454 are stored in the memory 41 in the form of computer programs executable in a computer. The processing circuitry 45 is a processor that reads the computer programs from the memory 41 and executes them so as to implement the function corresponding to each computer program.

In other words, after having read the computer programs, the processing circuitry 45 becomes equipped with the functions as illustrated in the processing circuitry 45 in FIG. 2.

With reference to FIG. 2, it is explained that a single unit of processing circuitry 45 is equipped with the processing functions implemented by the system control function 451, the preprocessing function 452, the reconstruction processing function 453, and the image processing function 454. Alternatively, the processing circuitry 45 can be configured by combining a plurality of independent processors, and each processor can be made to execute a computer program and implement the corresponding function.

In other words, either the configuration can be such that each function is configured as a function and a single unit of processing circuitry executes all computer programs, or the configuration can be such that specific functions are installed in program execution circuitry that is dedicated and independent in nature.

Based on an input operation received from the operator via the input interface 43, the system control function 451 controls various functions of the processing circuitry 45. For example, via the input interface 43, the system control function 451 receives input of user information (for example, the user ID) required for the login and receives input of subject information. Moreover, for example, the system control function 451 receives input of the imaging protocol via the input interface 43.

The preprocessing function 452 generates data as a result of performing preprocessing such as logarithmic conversion or offset correction, inter-channel sensitivity correction, and beam hardening correction with respect to the detection data output from the DAS 18. Herein, the data (detection data) before performing preprocessing and the data having been subjected to preprocessing is sometimes collectively referred to as projection data.

The reconstruction processing function 453 performs reconstruction processing with respect to the projection data, which is generated by the preprocessing function 452, using the filtered back projection method or the successive approximation reconstruction method according to the reconstruction conditions; and generates a plurality of sets of slice image data (CT image data).

The image processing function 454 implements a known method and, based on an input operation received from the operator via the input interface 43, converts the CT image data, which is generated by the reconstruction processing function 453, either into cross-sectional image data of an arbitrary cross-sectional surface or into three-dimensional image data. Meanwhile, alternatively, the three-dimensional image data can be directly generated by the reconstruction processing function 453.

The postprocessing can be performed either in the console apparatus 40 or in the medical image processing apparatus 2. Alternatively, the postprocessing can be simultaneously performed in the console apparatus 40 and the medical image processing apparatus 2.

The postprocessing defined herein represents the concept in which the processing is performed with respect to a plurality of sets of slice image data generated by the preprocessing function 452. For example, the postprocessing includes noise removal, multi-planar reconstruction (MPR) display of a plurality of sets of slice image data, and rendering of volume data.

Returning to the explanation with reference to FIG. 1, given below is the explanation about the medical image processing apparatus 2. The medical image processing apparatus 2 is an apparatus that, based on the sets of slice image data generated as a result of scanning the subject P using the X-ray CT apparatus 1, obtains an organ model of the target organ for treatment; estimates the feature quantity related to the post-treatment target organ; and displays the estimation result in the organ model.

In the embodiment, as an example, the explanation is given about a case in which, using a mitral valve mesh (an organ model) with respect to a patient suffering from mitral regurgitation, the feature quantities related to the shape of the mitral valve after performing edge-to-edge repair (for example, the valve area in the systole) is estimated, and the estimation result is displayed in the organ model.

As illustrated in FIG. 1, the medical image processing apparatus 2 includes, for example, a memory 21, a display 22, an input interface 23, and processing circuitry 24.

The memory 21 is used to store a variety of information. For example, the memory 21 is used to store setting information related to the display setting regarding the feature quantities. Moreover, for example, the memory 21 is used to store computer programs that enable the circuitry in the medical image processing apparatus 2 to implement the functions. Furthermore, for example, the memory 21 is used to store the data received from the X-ray CT apparatus 1 and to store the data generated by the processing circuitry 24.

The memory 21 is implemented, for example, using a semiconductor memory device such as a RAM or a flash memory, or using a hard disk, or using an optical disc. Alternatively, the memory 21 can be implemented using a server group (cloud) to which the medical image processing apparatus 2 is connected via the network NW.

The display 22 is used to display a variety of information. For example, the display 22 is used to display a GUI meant for receiving various instructions and settings from the user via the input interface 23. Moreover, the display 22 is used to display the organ model under the control performed by the processing circuitry 24. Furthermore, for example, the display 22 is used to display the estimated post-treatment feature quantities in the organ model.

The display 22 is a liquid crystal display or a CRT display. The display 22 can be of the desktop type or can be configured using a tablet terminal capable of performing wireless communication with the medical image processing apparatus 2.

The input interface 23 receives various input operations from the user, converts the received input operations into electrical signals, and outputs the electrical signals to the processing circuitry 24. For example, the input interface 23 is implemented using, for example, a mouse, a keyboard, a trackball, switches, buttons, a joystick, a touchpad on which an input operation is performed by touching the operation face, a touch-sensitive screen in which a display screen and a touchpad are integrated, contactless input circuitry in which an optical sensor is used, or a voice input circuit.

Alternatively, the input interface 23 can be configured using a tablet terminal capable of performing wireless communication with the main body of the medical image processing apparatus 2. Still alternatively, the input interface 23 can be configured as circuitry for receiving input operations from the user according to motion capturing. As an example, the input interface 23 can process the signals obtained via a tracker or images collected regarding the user, and can receive the body motion or the line of sight as the input operation.

Meanwhile, the input interface 23 is not limited to include a physical operating component such as a mouse or a keyboard. Alternatively, for example, the input interface 23 can be electric-signal processing circuitry that, from an external input apparatus installed independently from the medical image processing apparatus 2, receives an electric signal corresponding to an input operation, and outputs that electric signal to the processing circuitry 24.

The processing circuitry 24 executes a first acquisition function 241, a second acquisition function 242, a first setting function 243, a second setting function 244, an estimation function 245, a decision function 246, a determination function 247, and a display control function 248; and accordingly controls the operations of the entire medical image processing apparatus 2.

In the medical image processing apparatus 2 illustrated in FIG. 2, each processing function is stored as a computer-executable program in the memory 21. The processing circuitry 24 is a processor that reads computer programs from the memory 21 and executes them, and implements the functions corresponding to the computer programs. In other words, after having read the computer programs, the processing circuitry 24 becomes equipped with the functions corresponding to the read computer programs.

With reference to FIG. 1, it is explained that the first acquisition function 241, the second acquisition function 242, the first setting function 243, the second setting function 244, the estimation function 245, the decision function 246, the determination function 247, and the display control function 248 are implemented in a single unit of the processing circuitry 24. Alternatively, the processing circuitry 24 can be configured by combining a plurality of independent processors, and each processor can be made to execute a computer program and implement the corresponding function. Thus, the processing functions of the processing circuitry 24 can be implemented in a dispersed manner in a plurality of units of processing circuitry or in an integrated manner in a single unit of processing circuitry.

Alternatively, the processing circuitry 24 can implement the functions using the processor of an external apparatus that is connected via the network NW. For example, the processing circuitry 24 reads, from the memory 21, a computer program corresponding to each function and executes it; uses, as the calculation resource, the server group (cloud) to which the medical image processing apparatus 2 is connected via the network NW; and implements the functions illustrated in FIG. 1.

The first acquisition function 241 obtains medical images. For example, the first acquisition function 241 obtains, as medical images via the network NW, the slice image data generated when the X-ray CT apparatus 1 scans the subject P.

Meanwhile, if the medical information processing system S includes an image archiving apparatus, the first acquisition function 241 can obtain the slice image data from the image archiving apparatus such as a PACS server. For example, the first acquisition function 241 can monitor the PACS server via the network NW and, when a new set of slice image data is archived, can obtain that slice image data.

The second acquisition function 242 obtains an organ model in which the target organ is expressed.

For example, the second acquisition function 242 obtains the coordinate information of each pixel corresponding to the region of interest that represents the target region (organ) for treatment in the slice image data. More particularly, the second acquisition function 242 receives a specification input about the region of interest from the user and, according to the input, identifies the region of interest in the medical image. Then, based on the identified region of interest, the second acquisition function 242 obtains the coordinate information of each pixel corresponding to the region of interest.

The second acquisition function 242 can identify the region of interest based on the anatomical structure extracted from the slice image data according to a known region extraction method. Examples of the known region extraction method include the Otsu's method based on CT values, the region expansion method, the snake method, the graph cut method, and the mean shift method.

Alternatively, the second acquisition function 242 can identify the region of interest using a shape model generated using a known machine learning technology (including deep learning).

In this case, for example, the second acquisition function 242 applies, to the slice image data obtained by the first acquisition function 241, a shape model capable of extracting the coordinate information of a plurality of pixels corresponding to the region of interest in the image data. Then, the second acquisition function 242 identifies the region of interest based on the extraction result obtained as a result of applying the shape model.

The shape model is an already-learnt model in which, for example, a known machine learning technology (including a deep learning technology) is used and the relationship between the slice image data, in which the region of interest is captured and which is treated as input-side teacher data, and coordinate information of a plurality of pixels, which corresponds to the region of interest in the slice image data and which is treated as output-side teacher data, is learnt according to the dataset of the two types of teacher data.

Moreover, for example, the second acquisition function 242 can receive a selection input from the user about the region extraction method that, from among a plurality of region extraction methods, is to be implemented to perform region extraction, and can identify the region of interest according to the selected region extraction method. The identified region of interest is expressed using, for example, a three-dimensional mesh model.

In a three-dimensional mesh model, for example, a plurality of grid points is set on the identified region and the region of interest is expressed as a computational grid (hereinafter, called a mesh). In that case, the count and the arrangement of the grid points can be set in advance, or can be determined according to the size and the shape of the region of interest.

Meanwhile, the method for expressing the region of interest is not limited to the abovementioned method, and it is possible to implement any other method. For example, the region of interest can be expressed using a surface model.

The region of interest can be a two-dimensional region or a three-dimensional region. Alternatively, for example, if the first acquisition function 241 obtains four-dimensional images captured across a plurality of cardiac phases, the second acquisition function 242 can identify the region of interest in the image of each cardiac phase, and can obtain the images as a four-dimensional organ model including the information about the chronological morphology transformation in the target organ.

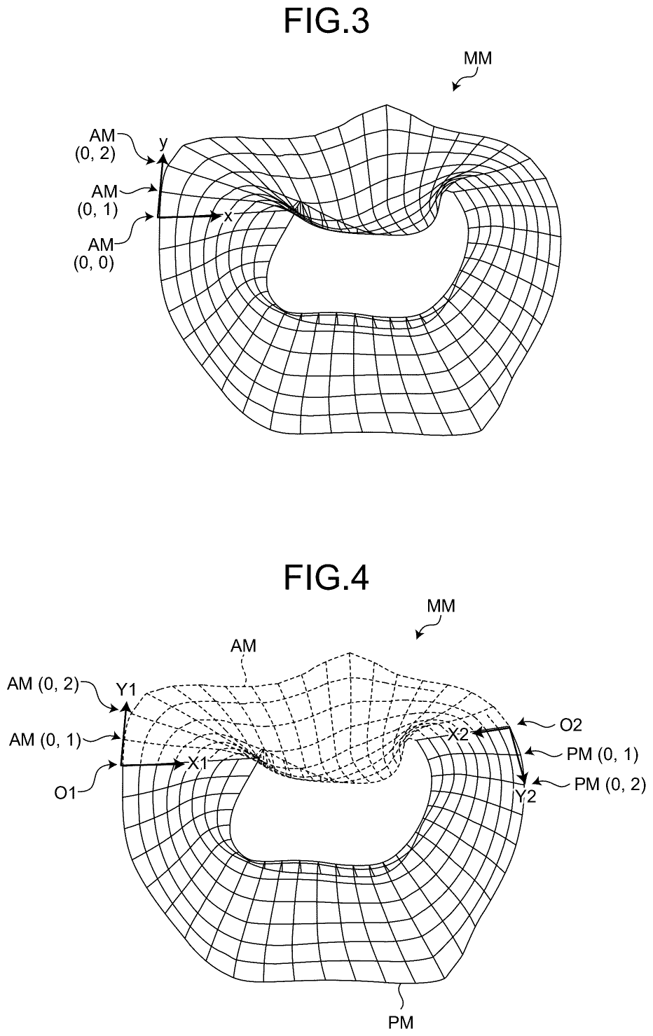

Given below is the explanation about the organ model with reference to a three-dimensional mesh model of the mitral valve (hereinafter, called a mitral valve mesh). FIG. 3 is a diagram illustrating an exemplary definition of a mitral valve mesh MM.

In the example illustrated in FIG. 3, regarding the direction from the valve ring side toward the valve leaflet side, a grid point group expressing the mitral valve is expressed in the X-coordinates having “0” positioned on the valve ring. Moreover, regarding the circumferential direction of the valve ring, the grid point group is expressed in the Y-coordinates having “0” positioned in between the anterior leaflet and the posterior leaflet. The mitral valve mesh MM illustrated in FIG. 3 expresses the mitral valve with 378 grid points in (9 rows)×(42 columns) positioned from (0, 0) to (8, 41) in the (X, Y) coordinate system.

When the region of interest is made of a plurality of structures, a different format can be specified for each structure in the organ model.

For example, since the mitral valve is made of the anterior leaflet and the posterior leaflet, the display color or the line style can be changed for the anterior leaflet and the posterior leaflet. Explained below with reference to FIG. 4 is the mitral valve mesh MM in the case in which the anterior leaflet and the posterior leaflet are set to have different formats. FIG. 4 is a diagram illustrating an exemplary definition of the mitral valve mesh MM. In the example illustrated in FIG. 4, the mitral valve mesh MM includes an anterior leaflet mesh AM and a posterior leaflet mesh PM.

The anterior leaflet mesh AM represents the region corresponding to the anterior leaflet of the mitral valve. In FIG. 4, the anterior leaflet mesh AM is made of 171 grid points in (9 rows)×(19 columns). In FIG. 4, a position O1 is treated as an origin (AM (0, 0)) of the anterior leaflet mesh AM. Moreover, in FIG. 4, the curve direction (the column direction) indicated by an arrow X1 is defined as the X-direction of the anterior leaflet mesh AM, and the curve direction (the row direction) indicated by an arrow Y1 is defined as the Y-direction of the anterior leaflet mesh AM.

The posterior leaflet mesh PM represents the region corresponding to the posterior leaflet of the mitral valve. In FIG. 4, the posterior leaflet mesh PM is made of 225 grid points in (9 rows)×(25 columns). Moreover, in FIG. 4, a position O2 is treated as the origin (PM (0, 0)) of the posterior leaflet mesh PM. Furthermore, in FIG. 4, the curve direction (the column direction) indicated by an arrow X2 is defined as the X-direction of the posterior leaflet mesh PM, and the curve direction (the row direction) indicated by an arrow Y2 is defined as the Y-direction of the posterior leaflet mesh PM.

In FIG. 4, the grid points (AM (0, 0) to AM (8, 0)) at one end of the anterior leaflet mesh AM and the grid points (PM (0, 2) to PM (8, 24)) at one end of the posterior leaflet mesh PM are common grid points. Moreover, the grid points (AM (0, 18) to AM (8, 18)) at the other end of the anterior leaflet mesh AM and the grid points (PM (0, 0) to PM (8, 0)) at the other end of the posterior leaflet mesh PM are common grid points. Hence, in an identical manner to the example illustrated in FIG. 3, the grid point group has 378 grid points in (9 rows)×(42 columns) across the mitral valve.

Returning to the explanation with reference to FIG. 1, the first setting function 243 sets first-type treatment conditions regarding the treatment method to be subjected to estimation. In the embodiment, the first-type treatment conditions represent the conditions that, from among the conditions used in the estimation operation performed by the estimation function 245 (explained later), are related to the treatment device.

For example, the first setting function 243 sets the size and the type of the treatment device to be used in the edge-to-edge repair of mitral regurgitation. The following explanation is given about the case in which the type of the treatment device is selected and set from among the following three types: MitraClip (registered trademark), PASCAL (by Edwards Lifesciences), and DragonFly (by Hangzou Valgen Medtech Co. Ltd.).

In this example, the display control function 248 (explained later) displays, in the display 22, a dropdown list that enables selection of one of the three types of treatment devices, namely, MitraClip (registered trademark), PASCAL (by Edwards Lifesciences), and DragonFly (by Hangzou Valgen Medtech Co. Ltd.). The first setting function 243 receives a selection input from the user about the type of the treatment device via the dropdown list, and sets the received type of the treatment device as the type of the treatment device.

Then, according to the set type of the treatment device, the display control function 248 displays, in the display 22, a dropdown list that enables selection of the size of the treatment device. The first setting function 243 receives a selection input about the size of the treatment device via the dropdown list, and sets the received size of the treatment device as the size of the treatment device.

As an example, when MitraClip (registered trademark) is set as the type of the treatment device, the display control function 248 displays, in the display 22, a dropdown list that enables selection between two clip widths of 4 mm and 6 mm and enables selection between two clip lengths of 9 mm and 12 mm.

Meanwhile, the first setting function 243 can obtain form information of the target organ from the organ model obtained by the second acquisition function 242; so that the type and the size of the organ device can be automatically set based on the form information. Examples of the form information include the distances or the angles among various feature points; the area, the volume, or the surface of the circumference of some or all of the region; and the degree of circularity or the degree of sphericity.

Moreover, in the embodiment, the first setting function 243 sets the depth and the angle of the treatment device to be used at the time of retaining the treatment device.

For example, after the size of the treatment device is set, according to the type and the size of the treatment device that are set, the display control function 248 displays, in the display 22, a dropdown list that enables selection of the depth and the angle of the treatment device.

The first setting function 243 receives a selection information about the depth and the angle of the treatment device via the dropdown list, and sets the received depth and the received angle of the treatment device as the depth and the angle of the treatment device.

As explained above, as a result of setting the depth and the angle of the treatment device, for example, when the treatment device to be retained is a clip, of the two places to be pinched by the treatment device, the position on one side can be uniquely identified from the position on the other side. More particularly, as a result of setting the position for pinching the anterior leaflet side of the mitral valve, it becomes possible to uniquely identify the position for pinching the posterior leaflet side.

Meanwhile, as a first-type treatment condition, the first setting function 243 can also set some other condition related to the treatment.

The second setting function 244 sets a second-type treatment condition of the target treatment method for estimation. In the embodiment, from among the conditions used in the estimation operation performed by the estimation function 245, the second-type treatment condition represents the condition related to the treatment position of the target organ.

The treatment position implies: the position at which the structure or the behavior of the target organ is altered as a result of performing treatment such as resection, needling, suture, or cauterization with respect to the target organ; or the position at which medication such as a curative medicine is administered or applied; or such a position in some part of the target organ which is related to the treatment action such as the retaining position or the retaining angle of the treatment device.

For example, in the edge-to-edge repair of mitral regurgitation, the second setting function 244 sets the retaining positions the treatment device (i.e., the position on the anterior leaflet side and the position on the posterior leaflet side of the mitral valve).

As an example, when the device to be retained is a clip, firstly, the second setting function 233 sets the position on the anterior leaflet side to be pinched using the treatment device. Then, according to the set position on the anterior leaflet side and according to the depth and the angle of the treatment device set as one of the first-type treatment conditions, the second setting function 244 identifies the position on the posterior leaflet side. Subsequently, the second setting function 244 sets the identified position on the posterior leaflet side as the position on the posterior leaflet side to be pinched using the treatment device.

In the case explained above, in the first-type treatment conditions (the type of the treatment device, the size of the treatment device, and the depth and the angle of the treatment device) set by the first setting function 243, the user becomes able to compare the difference in the estimated value of the post-treatment feature quantity depending on the difference in the retaining position of the treatment device. Thus, it can also be said that, since the effect of treatment can be estimated from the estimated value of the post-treatment feature quantity, the user becomes able to compare the difference in the estimated effect of treatment depending on the difference in the retaining position of the treatment device.

Meanwhile, instead of setting the depth and the angle of the treatment device, the first setting function 243 can set the position on the posterior leaflet side as a first-type treatment condition. In that case, the user becomes able to compare the difference in the estimated effect of treatment depending on the difference in the position on the anterior leaflet side at the position on the posterior leaflet side set by the first setting function 243.

Alternatively, the second setting function 244 can set the position on the posterior leaflet side or the depth and the angle of the treatment device as the second-type treatment conditions. Still alternatively, the second setting function 244 can set other treatment-related conditions other than the abovementioned conditions as the second-type treatment conditions.

In essence, as long as the first setting function 243 and the second setting function 244 set all treatment-related conditions to be used in the estimation operation performed by the estimation function 245, it does not matter which of the treatment-related functions are set by the first setting function 243 and by the second setting function 244.

For example, the second setting function 244 can receive, from the user via the user interface, a specification input about the grid points corresponding to the treatment position, and accordingly can set the treatment position.

Alternatively, for example, using the organ model displayed in an application by the display control function 248, the second setting function 244 can set the treatment position. In that case, the second setting function 244 receives, from the user, a selection input (a click) of the corresponding position on the displayed organ model and accordingly sets the treatment position.

Still alternatively, for example, without receiving any explicit specification from the user, the second setting function 244 can automatically set the treatment position based on the numbers assigned to the grid points. Still alternatively, the second setting function 244 can automatically set the treatment position based on the anatomical characteristics of the organ model.

Meanwhile, for example, in the case of repeatedly setting the treatment position for a plurality of number of times (in the case of setting a plurality of treatment positions), the second setting function 244 can perform the setting according to the user specification only for the first time; and, from the second time onward, can automatically set the treatment position based on the rules set in advance.

Based on the first-type treatment conditions and the second-type treatment conditions set by the first setting function 243 and the second setting function 244, respectively; the estimation function 245 performs the estimation operation for estimating the post-treatment state of the target organ. For example, the state to be estimated can be the form, the behavior, or the dynamics related to the target organ in part or in entirety; or can be the state of the fluids or the gases affected by the target organ; or can be the state related to the relationship between the target organ and the surrounding organs thereof, instead of the state of only the target organ.

Herein, it can be said that, since the effect of treatment can be estimated from the estimated value of the post-treatment feature quantity, the estimation function 245 estimates the effect of treatment of the target organ as carried out according to the first-type treatment conditions and the second-type treatment conditions.

Regarding the estimation method, as long as the actions of an object or the information related to fluids can be estimated, it is possible to implement any method. For example, the estimation function 245 implements a known method such as the finite element method, the finite difference method, or the immersed boundary method; and estimates the state of the post-treatment target organ.

The estimation function 245 can estimate the shape of the post-treatment target organ from a shape model built by learning the learning data that is prepared in advance using the machine learning technology including deep learning. In that case, for example, the shape model is an already-learnt model in which the mitral valve mesh obtained by the second acquisition function 242 and the first-type treatment conditions and the second-type treatment conditions set by the first setting function 243 and the second setting function 244, respectively, are treated as the input-side teacher data; the post-treatment shape of the target organ is treated as the output-side teacher data; and the relationship between the input-side teacher data and the output-side teacher data is learnt.

In the embodiment, with respect to each grid point of the organ model as obtained by the second acquisition function 242 with respect to the mitral valve, the estimation function 245 applies a mathematical model or a physical model that is set based on the first-type treatment conditions and the second-type treatment conditions set by the first setting function 243 and the second setting function 244, respectively.

Then, the estimation function 245 estimates the shape of the mitral valve after the retainment of the treatment device at the treatment position set as a second-type treatment condition by the second setting function 244. Moreover, from the estimated shape of the mitral valve, the estimation function 245 calculates the valve area as the estimated value of the post-treatment feature quantity.

Meanwhile, as the post-treatment feature quantity, the estimation function 245 can estimate the post-treatment blood flow information using the estimated post-treatment shape of the target organ. The blood flow information is, for example, the volume of regurgitant flow of the blood in the target organ. For example, based on a windkessel model or a pulse wave propagation model, an electrical circuitry model is designed in advance in which the circulatory dynamics of a biological body is simulated, and the estimation function 245 inputs the estimated post-treatment shape of the target organ to the electrical circuitry model. As a result, the estimation function 245 obtains the estimated post-treatment blood flow information.

More particularly, an electrical circuitry model is designed that enables calculation of the volume of regurgitant flow of the blood in the mitral valve based on the valve area of the mitral valve, and the estimation function 245 inputs the estimated post-treatment valve area to the electrical circuitry model. As a result, the estimation function 245 calculates the volume of regurgitant flow of the blood in the estimated post-treatment mitral valve.

Herein, the method for calculating the blood flow state is not limited to the method in which an electrical circuitry model is used as explained above. Alternatively, for example, the estimation function 245 can establish a system of equations of the Navier-Stokes equation with the necessary equations from among the equation of continuity, the Maxwell's equations, and the equation of state; can input various parameters to the equations; and can perform calculations to numerically obtain the required blood flow information.

Meanwhile, apart from the first-type treatment conditions and the second-type treatment conditions, the conditions required in the estimation operation can be set in advance, or can be automatically set according to the state of the organ model as obtained by the second acquisition function 242. Herein, the conditions include various parameters and environment conditions to be used in the estimation operation.

More particularly, as the parameters related to the leaflet, it is possible to enable setting of the hardness, the thickness, and the fiber direction of the valve. Moreover, as the parameters related to the tendinous cord, it is possible to enable setting of the hardness, the length, the thickness, the connecting positions, and the count. Furthermore, as the parameters related to the cardiac cavity or the blood vessels, it is possible to enable setting of the thickness, the volume, and the extent of smoothness of the surface of the blood vessels/the left atrium/the right atrium. Moreover, as the parameters related to the blood flow, it is possible to enable setting of the viscosity, the flow velocity, and the cuff pressure (the systolic blood pressure and the diastolic blood pressure) of the blood, and to enable setting of the total blood flow in the whole body.

Meanwhile, instead of directly setting each parameter, it can be made possible to set the parameters from indirect indexes based on a preset algorithm. For example, regarding the flow velocity of the blood passing through the mitral valve, the pressure gradient between the upper side (the left atrium side) and the lower side (the left ventricle side) of the mitral valve can be calculated based on the amount of change in the volume of the left atrium and the left ventricle in the time direction; and the flow velocity of the blood can be indirectly set from the pressure gradient. Thus, the volume of the left atrium and the left ventricle can be used as the parameter for setting the flow velocity.

The parameters given above are only exemplary, and the invention is not limited by the types of the parameters and the number of the parameters. Thus, the configuration can be such that, as long as the parameters are usable in the estimation operation for estimating the state of a biological body, any types of parameters are allowed to be set.

Regarding the parameters related to the clinical information mentioned in the electronic medical record, the estimation function 245 can identify the target information from the electronic medical record, the HIS, or the RIS connected to a network within the hospital; and can set the identified information.

The parameters to be used in the estimation operation by the estimation function 245 can be a mix of manually-set parameters and automatically-set parameters. Moreover, as the initial values of various parameters, the estimation function 245 can set predetermined constant numbers. The constant numbers can be set based on the attributes of the subject P. For example, the estimation function 245 can set different initial values according to the age and the gender of the subject P.

Meanwhile, the estimation function 245 need not set the condition that all predefined parameter items are to be used. For example, the estimation function 245 can receive, from the user via the user interface, a selection input about the parameter items to be used.

Moreover, for example, regarding the parameters obtained automatically due to image processing, the estimation function 245 can calculate the degree of reliability of the image processing. When the calculated degree of reliability exceeds a threshold value, the estimation function 245 sets the calculated values as the parameters. However, when the degree of reliability is equal to or smaller than the threshold value, the estimation function 245 can set predetermined constant numbers as the parameters, instead of setting the calculated values.

Moreover, for example, the estimation function 245 can prompt the user to manually set specific parameter items. If any parameter items are not manually set, the estimation function 245 can treat those parameter items as unused parameters.

Furthermore, for example, when the parameters are obtained from a different system as an electronic medical record and when those parameters are not specified in the concerned system, the estimation function 245 can set predetermined constant numbers as the parameters. Moreover, the estimation function 245 can prompt manual setting of such parameter items. Furthermore, the estimation function 245 can treat the unspecified parameter items as unused parameters.

Meanwhile, in addition to enabling setting of the parameters and the environment conditions of the target organ or the target patient, the estimation function 245 can also enable setting of calculation parameters to be used in performing the estimation operation. For example, the estimation function 245 can enable setting of the setting conditions for the computational grid (for example, the positions, the count, the shapes, and the types of elements (primary or secondary)) and can enable setting of convergence conditions (the step count (loop count) and the duration of the processing).

Based on the estimated state of the target organ, the decision function 246 decides on the display conditions regarding the treatment position in the organ model.

For example, based on the estimation result obtained by the estimation function 245, the decision function 246 decides on the display conditions regarding displaying, in the display 22, the organ model that is obtained by the second acquisition function 242. For example, the display conditions are meant for that position in the organ model which corresponds to the treatment position set by the second setting function 244.

In the embodiment, the decision function 246 sets the display conditions either for the positions of the actual grid points corresponding to the treatment position set by the second setting function 244 or for the positions surrounding those grid points. Meanwhile, the explanation given above is only exemplary, and the position in the organ model corresponding to the treatment position can be identified according to any method.

Meanwhile, for example, the display conditions can be color-related conditions (including coloring or grayscale) such as the color, the luminosity, the color saturation, and the permeability. Moreover, for example, when the positions of the grid points are illustrated using pictorial figures such as circles or using symbols, the display conditions can be conditions related to the shape of the pictorial figures or the symbols, such as the size and the shape of the symbols or the thickness of the outline. Alternatively, for example, when the organ model is illustrated in a mesh shape in which the grid points are joined by line segments, the display conditions can be the thickness and the color of the line segments that join the grid points. In essence, as long as the user is able to visually confirm the differences, the display conditions can be of any type.

Meanwhile, the decision function 246 either can set, in advance, the relationship between the estimated value of the feature quantity (the valve area or the volume of regurgitant flow) calculated by the estimation function 245 and the display conditions to be set, or can automatically decide on that relationship based on the distribution of the estimated values estimated at a plurality of treatment positions. For example, based on the maximum value and the minimum value from among the estimated values that are estimated at a plurality of treatment positions, the decision function 246 can decide on the display conditions (such as the color) corresponding to each estimated value.

After the decision function 246 decides on the display conditions, the determination function 247 determines whether or not the estimation operation is to be performed at another treatment position.

For example, after the decision function 246 has decided the display conditions, the display control function 248 displays, in the GUI, a message asking the user about whether or not to carry out the estimation operation at another treatment position using the estimation function 245, along with displaying a “Yes” button and a “No” button.

If an input indicating the pressing of the “Yes” button is received from the user, the determination function 247 determines that the estimation operation is to be performed at another treatment position. On the other hand, if an input indicating the pressing of the “No” button is received from the user, the determination function 247 determines that the estimation operation is not to be performed at another treatment position.

Alternatively, for example, based on the number of grid points in the organ model as obtained by the second acquisition function 242, the determination function 247 can determine whether or not the estimation operation is to be performed at another treatment position using the estimation function 245.

In that case, based on the number of grid points in the organ model as obtained by the second acquisition function 242, the determination function 247 identifies the treatment position for which the estimated value should be calculated in advance. In order to ensure that the setting operation for the treatment position, the decision operation for the display conditions, and the estimation operation are repeatedly performed until they get completed at all grid points corresponding to the concerned treatment position; the determination function 247 can determine whether or not the estimation operation is to be performed at another treatment position using the estimation function 245.

Herein, the determination function 247 either can perform the determination by treating all grid points as the treatment positions at which the estimated value should be calculated, or can identify the grid points satisfying specific conditions and can perform the determination by treating the identified grid points as the treatment positions at which the estimated value should be calculated.

For example, the specific conditions mentioned above can be those conditions among the first-type conditions set by the first setting function 243 which correspond to the positions at which treatment is possible. More particularly, it is a known fact that the length of the valve leaflet represents the applicable condition for the treatment device in the edge-to-edge repair. Hence, in order to ensure that the abovementioned operations are repeatedly performed until they get completed at the position at which the applicable condition is satisfied, the determination function 247 can determine whether or not the estimation operation is to be performed at another treatment position.

Moreover, for example, based on the relationship (the distance or the angle) between the commissure region or the annular area of the mitral valve and the characteristic position in the organ model, or based on the characteristic structure of the target organ such as the tendinous cord or based on the relationship with the surrounding organs such as the aortic valve, the determination function 247 can identify the grid points for which the estimated value should be calculated.

Furthermore, for example, in order to ensure that a new treatment position is set until the estimated value obtained by the estimation function 245 satisfies predetermined conditions (such as equal to or greater than a threshold value, equal to or smaller than a threshold value, or within a specific range), the determination function 247 can determine whether or not the estimation operation is to be performed at another treatment position.

The display control function 248 performs the control to display a variety of information in a display device. For example, according to the display conditions decided by the decision function 246, the display control function 248 displays the organ model, which is obtained by the second acquisition function 242, in the GUI displayed in the display 22.

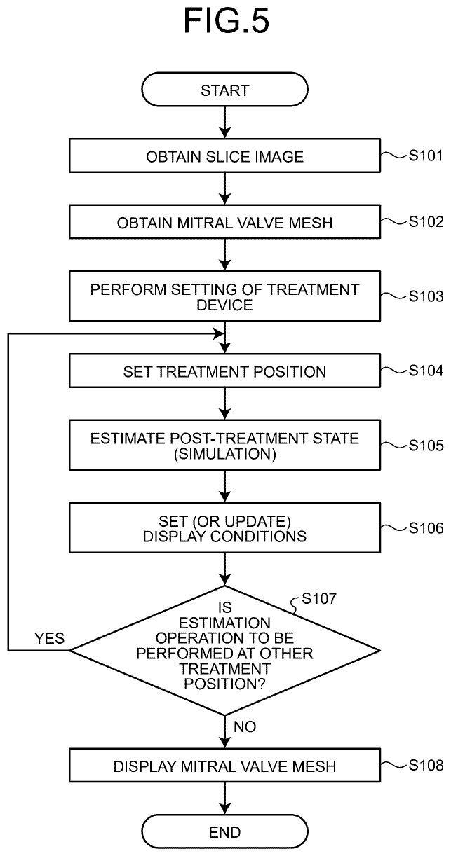

Given below is the explanation about the operations performed in the medical image processing apparatus 2 according to the embodiment. FIG. 5 is a flowchart for explaining an example of the operations performed in the medical image processing apparatus 2 according to the embodiment.

Firstly, the first acquisition function 241 obtains the slice image data (Step S101). For example, the first acquisition function 241 obtains the slice image data from the X-ray CT apparatus 1. In this example, the first acquisition function 241 obtains the slice image data in which the mitral valve during systole, when the mitral valve is closed, is illustrated.

Then, the second acquisition function 242 obtains the mitral valve mesh MM (organ model) from the slice image data (Step S102). For example, the second acquisition function 242 receives a specification input from the user about a region in the slice image data and, according to the specification input, extracts the mitral valve region from the slice image data. The second acquisition function 242 sets a plurality of grid points in the mitral valve region, and obtains the mitral valve mesh MM in which the mitral valve is expressed using the three-dimensional mesh model.

Subsequently, the first setting function 243 performs setting related to the treatment device (setting of the first-type treatment conditions) (Step S103). For example, according to the user input, the first setting function 243 selects and sets the treatment device from the three types, namely, MitraClip (registered trademark), PASCAL (by Edwards Lifesciences), and DragonFly (by Hangzou Valgen Medtech Co. Ltd.).

For example, when MitraClip (registered trademark) is decided as the type of the treatment device, according to the user input, the first setting function 243 selects and sets the clip width from two clip widths of 4 mm and 6 mm, and selects and sets the clip length from two clip lengths of 9mm and 12 mm. Moreover, for example, according to the user input, the first setting function 243 sets the depth and the angle of the treatment device.

Subsequently, the second setting function 244 performs setting related to the treatment position (setting of the second-type treatment conditions) (Step S104). For example, the second setting function 244 sets the position for pinching the anterior leaflet side and the position for pinching the posterior leaflet side of the mitral valve using the treatment device having the type and the size as set at Step S103.

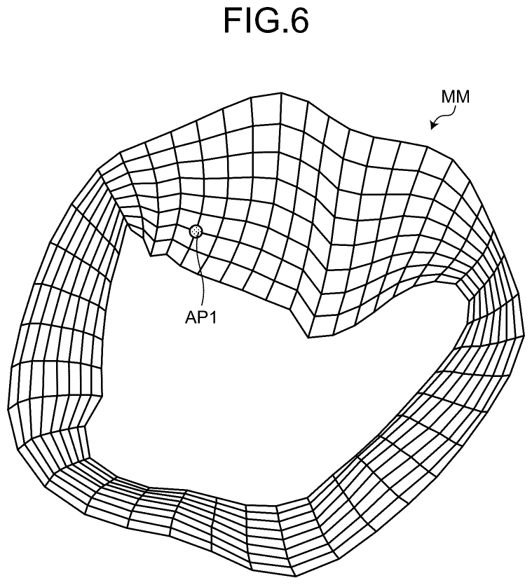

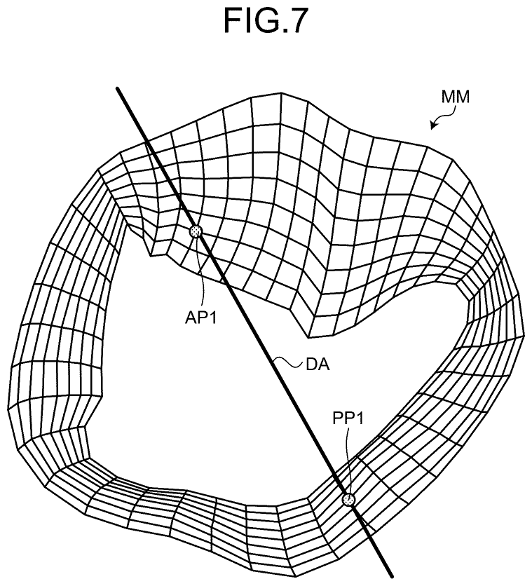

FIGS. 6 and 7 are diagrams for explaining an example of the setting operation for setting the treatment position. As illustrated in FIG. 6, the display control function 248 displays, in a GUI, the mitral valve mesh MM obtained at Step S102. The second setting function 244 receives, from the user, a specification input of a single grid point from among the grid points representing the anterior leaflet region of the mitral valve mesh MM. Then, the second setting function 244 sets the user-specified grid point as a position AP1 on the anterior leaflet side that is to be pinched using the treatment device.

As illustrated in FIG. 7, according to the set position AP1 and according to a retention angle DA of the device as decided from the depth and the angle of the treatment device as set at Step S103, the second setting function 244 identifies the grid point on the posterior leaflet side corresponding to the position AP1. Then, the second setting function 244 sets the identified grid point as a position PP1 on the posterior leaflet side that is to be pinched using the treatment device.

Returning to the explanation with reference to FIG. 5, after the operation at Step S104 is performed, the estimation function 245 estimates the state of the post-treatment mitral valve (Step S105). For example, the estimation function 245 implements a known method and estimates the shape of the mitral valve after the retainment of the treatment device at the treatment position set at Step S104. Then, from the estimated shape of the mitral valve, the estimation function 245 calculates the post-treatment estimated valve area.

Subsequently, the decision function 246 decides on the display conditions for the mitral valve mesh MM (Step S106). For example, based on the post-treatment valve area value estimated at Step S105, the decision function 246 sets the display condition of the grid points corresponding to the treatment position set at Step S104.

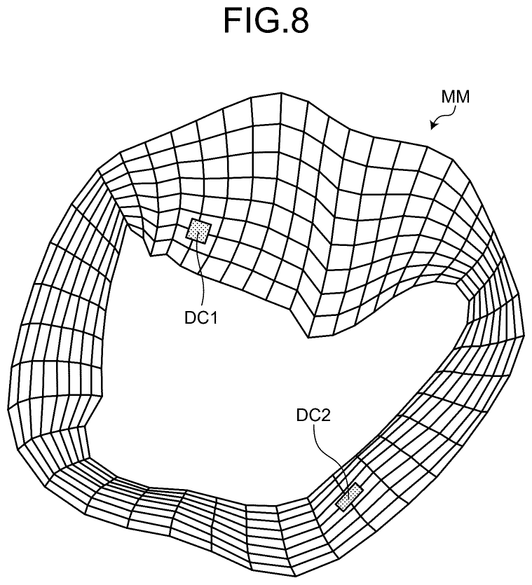

FIG. 8 is a diagram for explaining an example of the decision operation for deciding on the display conditions. As illustrated in FIG. 8, the decision function 246 sets the display conditions DC1 for the positions AP1 and the display conditions DC2 for the positions PP1, which are set at Step S104, according to the post-treatment estimated valve area value that is estimated at Step S105.

More particularly, the decision function 246 decides on the display conditions according to the following rules: when the estimated valve area value is between XX mm2 and YY mm2,the display is to be done in blue color; when the estimated valve area value is between YY+1 mm2 and ZZ mm2, the display is to be done in yellow color; and when the estimated value area value is between ZZ+1 mm2 and AA mm2, the display is to be dome in red color. In the example illustrated in FIG. 8, the decision function 246 can apply different display conditions for the position AP1 and for the position PP1. For example, the decision function 246 can set the permeability of the display of the position PP1 to be lower than the permeability of the display of the position AP1.

Returning to the explanation with reference to FIG. 5, after the operation at Step S106 is performed, the determination function determines whether the estimation operation is to be performed at another treatment position (Step S107). For example, according to an instruction from the user, the determination function 247 determines whether the estimation operation is to be performed at another treatment position.

If the estimation operation is to be performed at another treatment position (Yes at Step S107), the system control returns to Step S103.

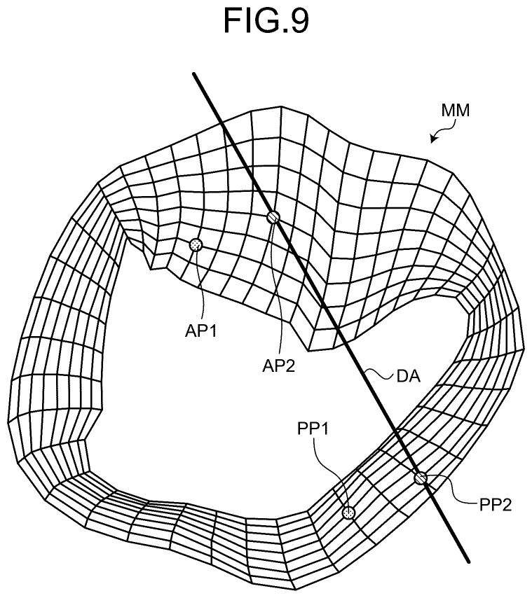

FIG. 9 is a diagram for explaining an example of an addition setting operation for adding a treatment position. As illustrated in FIG. 9, in an identical sequence to the sequence illustrated in FIGS. 6 and 7, the second setting function 244 sets another position AP2 on the anterior leaflet side and another position PP2 on the posterior leaflet side that are to be pinched using the treatment device.

When the second setting function 244 performs the setting operation for another treatment position, the decision function 246 performs an updating operation for updating the display conditions about the organ model.

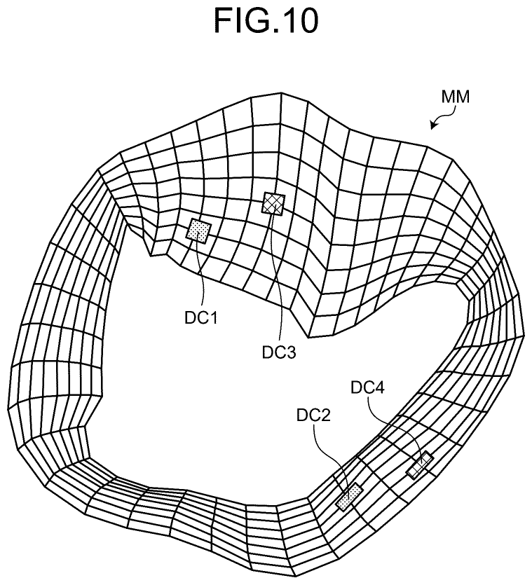

FIG. 10 is a diagram for example of the updating operation for updating the display conditions. As illustrated in FIG. 10, according to the post-treatment estimated valve area value at the other treatment position set by the second setting function 244, the decision function 246 updates the display conditions for the mitral valve mesh MM.

For example, according to the difference between the estimated valve area value when the positions AP1 and PP1 are pinched (hereinafter, also referred to as an estimated value EV1) and the estimated valve area value when the positions AP2 and PP2 are pinched (hereinafter, also reference to an estimated value EV2) (i.e., when three or more treatment positions are set, according to the difference between the maximum value and the minimum value of the estimated valve area values), the decision function 246 decides on the display condition DC1 for the position AP1, the display condition DC2 for the position PP1, the display condition DC3 for the position AP2, and the display condition DC4 for the position PP2 and thus updates the display conditions of the mitral valve mesh MM.

As an example, when the predetermined rules indicate that the estimated values EV1 and EV2 have blue color as the display color and that the estimated value EV1 is greater than the estimated value EV2, the decision function 246 can update the display conditions by modifying the predetermined rules in such a way the positions AP1 and PP1 are displayed in yellow color. As a result, it becomes easier for the user to understand the difference in the estimated effects of treatment that is attributed to the difference in the treatment positions.

Returning to the explanation with reference to FIG. 5, when the estimation operation is not to be performed at Step S107 (No at Step S107), according to the display conditions decided or updated at Step S106, the display control function 248 displays the mitral valve mesh MM, which is obtained at Step S102, in the GUI (Step S108). That marks the end of the operations.

Meanwhile, at Step S106, the decision function 246 can decide on the display conditions based on the estimated valve area values at a plurality of treatment positions. In that case, the sequence of the operations performed at Steps S106 and S107 is reversed.

Meanwhile, after the operation at Step S106 is performed, the system control can proceed to Step S108 without proceeding to Step S107.

Herein, it can be said that the position on the anterior leaflet side and the position on the posterior leaflet side that are pinched using the treatment device and that are set at Step S104 represent the treatment positions, and it can be said that the post-treatment valve area value estimated at Step S105 represents the effect of treatment. Hence, it can be said that, at Step S108 in the example explained above, the display control function 248 displays, in the organ model of the target organ, the information indicating the relationship between the user-specified treatment positions and the effect of treatment presumed when treatment is performed at those treatment positions.

Meanwhile, in the example explained above, after Step S108, if another treatment position is additionally specified by the user, the display control function 248 can reset the information indicating the relationship between the currently-specified treatment position in the organ model of the target organ and the effect of treatment estimated when the treatment is performed at the currently-specified treatment position. In that case, the display control function 248 can display the information indicating the relationship between the post-resetting other treatment position and the effect of treatment presumed when the treatment is performed at that other treatment position.

Moreover, the display control function 248 can display the following organ models side-by-side: the organ model that expresses the information indicating the relationship between the currently-specified treatment position and the effect of treatment with respect to the currently-specified treatment position, and the organ model that expresses the information indicating another treatment position and the effect of treatment with respect to the other treatment position.

The medical image processing apparatus 2 according to the embodiment described above obtains an organ model in which the target organ for treatment is expressed; identifies the treatment conditions including the treatment position in the organ model; estimates the post-treatment feature quantity based on the organ model and the identified treatment conditions; decides on the display conditions regarding the treatment position in the organ model based on the estimated feature quantity; and displays the organ model in the display 22 according to the decided display conditions.

As a result, according to the feature quantity estimated from the treatment conditions including the treatment position, the medical image processing apparatus 2 according to the embodiment can modify the display form at that position in the organ model representing the pre-treatment state which corresponds to the treatment position. Thus, in the organ model representing the pre-treatment state, the user becomes able to confirm the relationship between the treatment position and the estimated value of the post-treatment feature quantity at that treatment position. Since the effect of treatment can be estimated from the estimated value of the post-treatment feature quantity, in the medical image processing apparatus 2 according to the embodiment, it becomes easier for the user to understand the relationship between the pre-treatment state, the treatment conditions, and the effect of treatment estimated under those treatment conditions.

Meanwhile, the embodiment described above can be appropriately modified by varying some of the configurations or the functions of the apparatuses. Given below is the explanation of modification examples of the embodiment as other embodiments. In the following explanation, the focus is on explaining the differences with the embodiment described above. Thus, regarding the same details as the details already explained above, the detailed explanation is not given again. The modification examples explained below can be implemented either individually or in combination.

### First Modification Example

In the embodiment described above, the explanation is given about the case in which a CT image obtained from the X-ray CT apparatus 1 represents a medical image. However, that is not the only possible case. Alternatively, for example, a medical image can be an image obtained from a medical image diagnostic apparatus such as an MRI apparatus (MRI stands for Magnetic Resonance Imaging), an angio CT system, a tomosynthesis apparatus, a SPECT apparatus (SPECT stands for Single Photon Emission Computed Tomography), a PET apparatus (PET stands for Positron Emission computed Tomography), or an ultrasonic diagnostic apparatus.

Moreover, regarding the type of medical images, as long as the form information of the target organ is stored therein, the medical images can be on any type. Thus, a medical image can be a three-dimensional image or a two-dimensional image, or can be a four-dimensional image obtained by capturing three-dimensional images or two-dimensional images in the time direction.

According to a first modification example, based on the medical images other than CT images, it becomes possible to enable easy understanding of the relationship between the treatment conditions and the effect of treatment estimated under those treatment conditions.

### Second Modification Example

In the embodiment described above, the explanation is given about the case in which the treatment method indicates treating the mitral valve using a treatment device of the edge-to-edge repair type. However, the target organ and the treatment method (for example, the treatment device and the procedure) are not limited to that example.

For example, the present method can be implemented in transcatheter aortic valve implantation (TAVI) for treating aortic regurgitation. Explained below with reference to FIG. 5 is an example of implementing the present method in transcatheter aortic valve implantation for treating aortic regurgitation.

For example, at Step S101 illustrated in FIG. 5, the first acquisition function 241 obtains a medical image in which the aortic valve, the ascending aorta, and the left ventricular outflow tract (LVOT) are captured. Moreover, for example, at Step S102, the second acquisition function 242 obtains an organ model in which the aortic valve, the ascending aorta, and the left ventricular outflow tract (LVOT) are expressed.

Furthermore, for example, at Step S103, the first setting function 243 sets the size and the type of the treatment device. Moreover, for example, at Step S104, the second setting function 244 sets the retaining position (for example, the depth) of the treatment device.

Meanwhile, instead of setting the depth of the treatment device, the second setting function 244 can set the retaining angle of the treatment device. Moreover, the second setting function 244 can set, as a condition about the treatment position, the straight-line distance from the coronary ostium to the front end of the treatment device.

Moreover, for example, at Step S105, the estimation function 245 estimates the blood flow rate in the aortic valve or estimates the blood flow rate toward the coronary artery. Furthermore, for example, at Step S106, the decision function 246 decides on the display condition according to the estimated blood flow rate toward the coronary artery.

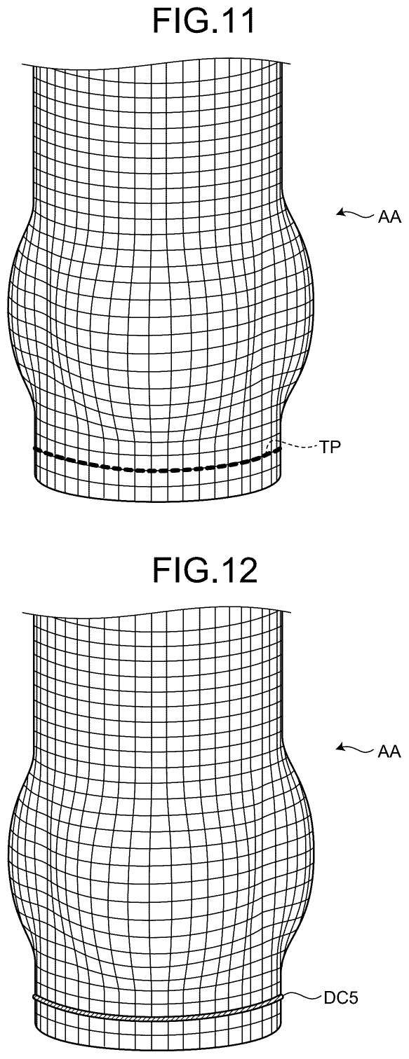

FIGS. 11 to 13 are diagrams for explaining an example of setting the display conditions of the organ model according to the second modification example. In FIG. 11 is illustrated an example of a treatment position TP that is set in a three-dimensional mesh model AA including the region near the aorta (hereinafter, also called an aortic valve mesh AA). In that case, the treatment position TP expresses the front end position of the treatment device (a prosthetic valve).

In this example, as the post-treatment feature quantity, the estimation function 245 estimates the blood flow rate toward the coronary artery in the case in which a prosthetic valve is retained to have its front end position at the already-set treatment position. As illustrated in FIG. 12, the decision function 246 decides on a display condition DC5 at the treatment position according to the estimated blood flow rate toward the coronary artery. Herein, since the front end of the prosthetic value is circular, all grid points in one circle corresponding to the front end position of the prosthetic valve in the coronary aortic valve mesh AA have the same display condition.

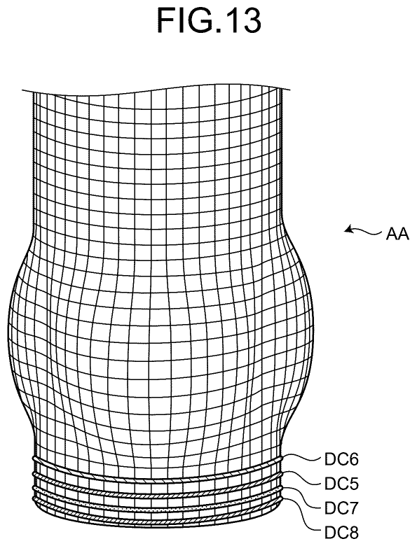

Meanwhile, if the front end positions of a plurality of prosthetic valves is set and the operations identical the operations explained earlier are performed, as illustrated by display conditions DC5 to DC8 in FIG. 13, the user becomes able to visually understand the difference in the estimated values of the blood flow rate toward the coronary artery thar is attributed to the difference in the retaining positions the prosthetic valve. Moreover, from the blood flow rate toward the coronary artery, the user becomes able to estimate the effect of treatment. For that reason, when the present method is implemented in transcatheter aortic valve implantation for treating aortic regurgitation, the user becomes able to easily understand the difference in the estimated effects of treatment that is attributed to the difference in the retaining positions of the prosthetic valve.

Meanwhile, for example, the present method can be implemented in performing the cerebral aneurysm clipping surgery against cerebral aneurysm. The cerebral aneurysm clipping surgery is done to prevent an unruptured cerebral aneurysm from rupturing (from progressing to subarachnoid hemorrhage). Explained below with reference to FIG. 5 is an example of implementing the cerebral aneurysm clipping surgery against cerebral aneurysm.

For example, at Step S101 illustrated in FIG. 5, the first acquisition function 241 obtains a medical image in which that artery in the brain of the subject P is captured which includes a portion having cerebral aneurism. Moreover, for example, at Step S102, the second acquisition function 242 obtains an organ model in which the artery of the brain is expressed.

Furthermore, for example, at Step S103, the first setting function 243 sets the size or the type of the treatment device (a clip). Moreover, for example, at Step S104, the second setting function 244 sets the retaining position and the retaining angle of the clip.

Furthermore, for example, at Step S105, the estimation function 245 estimates the blood flow rate toward the cerebral aneurism. Moreover, for example, at Step S106, the decision function 246 decides on the display conditions according to the estimated blood flow rate toward the cerebral aneurism.

In the example explained above, the user becomes able to visually understand the difference in the estimated values of the blood flow rate toward the cerebral aneurism that is attributed to the difference in the retaining positions and the retaining angles of the clip. Moreover, from the blood flow rate toward the cerebral aneurism, the user becomes able to estimate the effect of treatment. For that reason, when the present method is implemented in performing the cerebral aneurysm clipping surgery against cerebral aneurysm, the user becomes able to easily understand the difference in the estimated effects of treatment that is attributed to the difference in the retaining positions and the retaining angles of the clip.

### Third Modification Example

In the embodiment described earlier, the explanation is given about the case in which the second setting function 244 sets the treatment conditions for a single treatment position. In a third modification example, the explanation is given about the case in which it is made possible to set some other conditions in addition to setting the conditions for the treatment position. Explained below with reference to FIG. 5 is an example in which the second setting function 244 sets two types of treatment conditions, namely, the treatment position and the device size.

For example, at Step S104, the second setting function 244 enables setting two types of treatment conditions, namely, the treatment position and the device size. Moreover, for example, at Step S105, the estimation function 245 estimates the post-treatment state based on the two types of treatment conditions that are set. Furthermore, for example, in the third modification example, the operation at Step S107 is performed before performing the operation at Step S106.

In that case, at Step S107, the determination function 247 determines, at the same treatment position, whether or not the post-treatment state is to be estimated for a different device size. When the operation at Step S103 is performed in a repeated manner and a plurality of device sizes is set as a treatment condition, the second setting function 244 can perform the operation at Step S105 in a repeated manner and can estimate, at the same treatment position, the post-treatment state regarding a plurality of device sizes.

Moreover, for example, at Step S106, the decision function 246 calculates the difference in the results obtained due to different device sizes at the same treatment position. In that case, according to the magnitude of the differences in the calculated results, the decision function 246 modifies the display conditions.

As an example, the decision function 246 sets the permeability to a lower level when the difference is small, and sets the permeability to a higher level when the difference is large. As a result, it becomes easier for the user to identify the positions at which the effect of the differences in the device size is large or small.

The explanation given above is about the example in which the device size is set as a treatment condition along with the treatment condition indicating the treatment position. However, the treatment condition that is set along with the treatment position by the second setting function 244 is not limited to the device size. Moreover, the number of types of treatment conditions is also not limited to two. For example, the second setting function 244 can set three or more types of treatment conditions.

However, in the case explained above, regarding a plurality of treatment conditions, in order to enable visual confirmation of the difference in the display conditions, the decision function 246 needs to decide on different display conditions. For example, apart from using the permeability, the decision function 246 can use the luminosity, the color saturation, the frame thickness, the frame color, and the frame shape; and express the difference among the treatment conditions. Meanwhile, in the third modification example, the second setting function 244 needs to set the treatment conditions regarding at least one position in the target organ.

According to the third modification example, for example, regarding each of plurality of types of treatment conditions, the user becomes able to visually understand the difference in the concerned treatment condition at the same treatment position.

### Fourth Modification Example

In the embodiment described earlier, the explanation is given about the case in which the decision function 246 decides on the display conditions according to the estimated value of the post-treatment feature quantity. In a fourth modification example, the explanation is given about a case in which the decision function 246 corrects the display conditions using information other than the estimated value obtained by the estimation function 245.

The information other than the estimated value indicates, for example, the position information in the target organ, or the information regarding the relationship with the surrounding organs, or the information about the attributes of the user or the patient. Moreover, for example, correction of the display conditions indicates setting the display conditions based on the estimated value of the feature quantity as obtained by the estimation function 245 as well as based on the information other than the estimated value, or indicates modifying or correcting the display conditions, which are set based on the estimated value of the feature quantity, based on the information other than the estimated value.

As an example, the decision function 246 evaluates, either automatically or manually, the ease of retaining the treatment device in the target organ. The decision function 246 corrects the display condition using the evaluation result. More particularly, if the user evaluates that the treatment device is easier to retain near the center of the valve leaflet than near the commissure region of the mitral valve, the decision function 246 can decide on the display conditions in such a way that the permeability is lower near the center of the valve leaflet than near the commissure region of the mitral valve.

As a result, for example, the user becomes able to visually confirm the target organ model under the display conditions in which the ease of retainment of the treatment device and the estimated value of the post-treatment feature quantity are taken into account. For that reason, it becomes easier for the user to examine the position at which the treatment device should be retained.

Moreover, for example, the decision function 246 can identify the position of the tendinous cord according to a known method, and can correct the display conditions according to the distance from the identified position of the tendinous cord. Alternatively, the decision function 246 can set, in advance, the favorable retaining positions and the unfavorable retaining positions for the user, and can correct the display conditions based on those positions. Still alternatively, the decision function 246 can correct the display conditions according to the age, the medication history, or the treatment history of the concerned patient.

Still alternatively, for example, the decision function 246 can correct the display conditions based on the estimated value of the post-treatment feature quantity at a plurality of treatment positions. As an example, when adjacent grid points are set as the treatment positions, if a predetermined condition, such as obtaining the estimated values of mutually similar feature quantities, is fulfilled; the decision function 246 can correct the display conditions.

In the explanation given above, the case in which the estimated values of mutually similar feature quantities are obtained is treated as the predetermined condition. Alternatively, the case in which the estimated values of mutually high feature quantities are obtained or the case in which the estimated values of mutually low feature quantities are obtained can be treated as the predetermined condition. Moreover, when the grid points fulfilling the predetermined condition are consecutive, the decision function 246 can correct the display conditions according to the number of consecutive grid points.

### Fifth Modification Example

In the embodiment described above, the explanation is given about the case in which the estimation function 245 estimates a single feature quantity. In a fifth modification example, the explanation is given about the case in which the estimation function 245 estimates a plurality of feature quantities. For example, regarding two feature quantities, namely, the valve area and the volume of regurgitant flow, the estimation function 245 can estimate the post-treatment feature quantity.

When the estimated values of a plurality of feature quantities are obtained, for example, the decision function 246 can additionally perform the operation of calculating a single estimating value by integrating the estimated values of the feature quantities as well as can set the display conditions based on the integrated estimated value.

Alternatively, for example, the decision function 246 can set different display conditions for the estimated value of each of a plurality of feature quantities. As an example, the decision function 246 can express the estimated value of the valve area using a color, and can express the estimated value of the volume of regurgitant flow using the permeability.

According to the fifth modification example, the treatment condition can be examined by taking into account a plurality of feature quantities. Moreover, according to the fifth modification example, the estimated value of each of a plurality of feature quantities can be associated to a different display condition, thereby making it easier for the user to visually confirm the difference among the estimated values of a plurality of feature quantities that is attributed to the difference in the treatment positions.

### Sixth Modification Example

In the embodiment described above, the explanation is given about the case in which, the display control function 248 does not perform control to display the treatment conditions that are related to the treatment device and that are set by the first setting function 243 at Step S103 illustrated in FIG. 5. In a sixth modification example, the explanation is given about the case in which the display control function 248 performs control to display the treatment conditions that are related to the treatment device and that are set by the first setting function 243.

For example, at the time of displaying the organ model in the display 22 at Step S108 illustrated in FIG. 5, the display control function 248 can perform control to display the treatment conditions, which are set by the first setting function 243, near the organ model.

At Step S103 illustrated in FIG. 5, the treatment conditions related to the treatment device set by the first setting function 243 are not expressed as the display conditions of the target organ model. For that reason, when the treatment conditions related to the treatment device are displayed closely as characters or numerical values, it becomes possible to further promote the understanding of the user.

### Seventh Modification Example

In the embodiment described above, the explanation is given about the case in which the medical image processing apparatus 2 includes the first acquisition function 241, the second acquisition function 242, the first setting function 243, the second setting function 244, the estimation function 245, the decision function 246, the determination function 247, and the display control function 248. However, some or all of those functions can be provided in a different device.

For example, the console apparatus 40 of the X-ray CT apparatus 1 can include the first acquisition function 241, the second acquisition function 242, the first setting function 243, the second setting function 244, the estimation function 245, the decision function 246, the determination function 247, and the display control function 248.

Alternatively, for example, the medical information processing system S can further include another apparatus such as a workstation other than the medical image processing apparatus 2, and that workstation can include some functions from among the first acquisition function 241, the second acquisition function 242, the first setting function 243, the second setting function 244, the estimation function 245, the decision function 246, the determination function 247, and the display control function 248.

As a result, the medical information processing system S according to the seventh modification example can club the abovementioned functions in a single apparatus or can disperse the abovementioned function across a plurality of apparatuses. Hence, the medical information processing system S according to the seventh modification example can adapt the system configuration according to the user environment.

According to the embodiment and the modification examples described above, regarding the relationship between the pre-treatment state, the treatment conditions, and the effect of treatment estimated under those treatment conditions, it becomes possible to make the understanding easier.

The term “processor” mentioned above implies, for example, a central processing unit (CPU), a graphics processing unit (GPU), or circuitry such as an application specific integrated circuit (ASIC) or a programmable logic device (for example, a simple programmable logic device (SPLD), a complex programmable logic device (CPLD), or a field programmable gate array (FPGA)).

A processor reads computer programs stored in the memory 41, and executes them to implement the functions. Meanwhile, instead of storing the computer programs in the memory 41, they can be directly embedded in the circuitry of the processor. In that case, the processor reads the computer programs embedded in the circuitry, and executes them to implement the functions.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A medical image processing apparatus comprising processing circuitry that
obtains an organ model, in which a target organ for treatment is expressed, based on a medical image,
identifies a treatment condition which represents a treatment-related condition including at least treatment position in the organ model,
estimates a post-treatment feature quantity based on the organ model and the treatment condition,
decides on a display condition regarding the treatment position in the organ model based on the estimated feature quantity, and
displays, in a display device, the organ model according to the decided display condition.

2. The medical image processing apparatus according to claim 1, wherein the processing circuitry
identifies the treatment condition including a plurality of the treatment position,
estimates the feature quantity at each of the plurality of treatment positions, and
decides on a display condition regarding the treatment positions in the organ model based on the plurality of estimated feature quantities.

3. The medical image processing apparatus according to claim 1, wherein, from a user, the processing circuitry receives an input specifying a region in the organ model and identifies the treatment position based on the input.

4. The medical image processing apparatus according to claim 3, wherein
the treatment condition includes a device condition related to a treatment device that is involved in treatment of the organ, and
the processing circuitry
receives, from a user, an input specifying the device condition,
identifies the device condition based on the input, and
identifies the treatment position based on the input and the identified device condition.

5. The medical image processing apparatus according to claim 4, wherein the processing circuitry evaluates ease of retainment of the treatment device and, based on evaluation result, corrects the decided display condition.

6. The medical image processing apparatus according to claim 4, wherein the processing circuitry displays, in the display device, the organ model and information indicating the identified device condition.

7. The medical image processing apparatus according to claim 1, wherein
the processing circuitry further includes a calculating unit that
estimates a plurality of types of the feature quantity, and
calculates integrated feature quantity by integrating the plurality of estimated types of feature quantity, and
the deciding unit decides on the display condition based on the integrated feature quantity that is calculated.

8. The medical image processing apparatus according to claim 1, wherein the display condition includes at least one of conditions related to color, luminosity, color saturation, permeability, thickness of a frame in which the treatment position is expressed, color of the frame, and shape of the frame.

9. The medical image processing apparatus according to claim 8, wherein the processing circuitry
estimates a plurality of types of the feature quantity, and
for each of the types of feature quantity, decides on the display condition of a different type.

10. The medical image processing apparatus according to claim 1, wherein the processing circuitry
receives, from a user, an input about information indicating the treatment position,
identifies the treatment position based on the received information indicating the treatment position,
estimates, as effect of treatment in case of performing treatment at the treatment position, the post-treatment feature quantity in case of performing treatment at the identified treatment position,
decides on the display condition based on extent of the estimated effect of treatment, and
displays, in the organ model, information indicating relationship between the treatment position and the effect of treatment.

11. The medical image processing apparatus according to claim 10, wherein the processing circuitry
receives input of information indicating a plurality of the treatment position, and
decides on the display condition in such a way that the effect of treatment at each of the plurality of treatment positions is distinguishable.

12. The medical image processing apparatus according to claim 1, wherein the processing circuitry
obtains the organ model in which mitral valve is expressed,
identifies the treatment condition that at least includes
type and size of a mitral valve repairing device used in performing edge-to-edge repair in which a synapsed region is increased by grasping anterior leaflet and posterior leaflet of the mitral valve, and
positions of the anterior leaflet and the posterior leaflet that represent the treatment position and that are grasped using the mitral valve repairing device, and
estimates the feature quantity regarding shape of the mitral valve after the anterior leaflet and the posterior leaflet are grasped using the mitral valve repair device.

13. A medical image processing method implemented in a medical image processing apparatus, comprising:
obtaining an organ model, in which a target organ for treatment is expressed, based on a medical image;
identifying a treatment condition which represents a treatment-related condition including at least treatment position in the organ model;
estimating a post-treatment feature quantity based on the organ model and the treatment condition;
deciding on a display condition regarding the treatment position in the organ model based on the estimated feature quantity; and
displaying, in a display device, the organ model according to the decided display condition.

14. A non-transitory computer-readable storage medium having a computer program stored therein, wherein the computer program
obtains an organ model, in which a target organ for treatment is expressed, based on a medical image,
identifies a treatment condition which represents a treatment-related condition including at least treatment position in the organ model,
estimates a post-treatment feature quantity based on the organ model and the treatment condition,
decides on a display condition regarding the treatment position in the organ model based on the estimated feature quantity, and
displays, in a display device, the organ model according to the decided display condition.
